(19) United States
(12) Patent Application Publication
(10) Pub. No.: US 2006/0129068 A1
(43) Pub. Date: Jun. 15, 2006

(54) Palpometer and methods of use thereof

(76) Inventors: Arthur Makosinski, Victoria (CA); Adam Zielinski, Victoria (CA); Christopher Atkins, Victoria (CA)

Correspondence Address: KLARQUIST SPARKMAN, LLP, 121 SW SALMON STREET, SUITE 1600, PORTLAND, OR, 97204, US

(21) Appl. No.: 10/535,541

(22) PCT Filed: Nov. 18, 2003
(86) PCT No.: PCT/CA03/01782

§ 371 Date: Nov. 21, 2005

Related U.S. Application Data
(60) Provisional application No. 60/427,486, filed on Nov. 18, 2002.

(51) Int. Cl. A61B 5/103

(52) U.S. Cl. 600/587

(57) ABSTRACT

Dolorimeters and methods of use thereof are provided. The dolorimeters comprise an integral apparatus having sensor means having a property which varies according to pressure or force applied to the means and a reporter means. The dolorimeter can be worn on a finger or thumb of a practitioner and allows for quantification of pressure or force applied to a patient. The reporter may send an audible, light or radio signal. The dolorimeter is further provided with means for calibration and an initiator to initiate calibration.

### BACKGROUND OF THE INVENTION

1. Field of the Invention

This invention relates to an apparatus for measuring pressure applied to a patient in order to assess discomfort and more specifically to a digit-mounted integral unit for assessing pressure, for example the pressure applied to a joint of an arthritic patient.

2. Description of the Prior Art

Physicians place an important role on patterns of pain in the diagnosis and management of their patients. Manual palpitation is the standard method of examination, but it has a certain drawbacks, namely that the procedure is subjective and lacks the precision necessary to accurately assess, for example, the degree of inflammation of arthritic patients.

The limitations of manual palpitation have been addressed by providing mechanical devices known as dolorimeters, algesimeters or algometers (the terms are used synonymously herein). In the simplest form, a mechanical dolorimeter includes a simple spring loaded probe connected to a gauge. The gauge indicates the deflection of the probe and hence the pressure applied to the probe. In use, the physician presses the probe against the inflamed joint or other portion of the patient's body suffering pain, and applies pressure until the patient feels discomfort. The reading of the gauge is noted, the reading being an objective indication of the degree of inflammation of the joint, for example. Electronic dolorimeters have been developed, such as disclosed in U.S. Pat No. 4,641,661, incorporated herein by reference. This device includes an electronic circuit housed in a hand-held unit. The dolorimeter has a probe with a resistance that varies according to pressure applied to the probe. The hand-held unit is capable of measuring the resistance of the probe and thereby the pressure applied.

Other devices for determining or recording the pain sensitivity or the like are disclosed in U.S. Pat. No. 4,501,148, incorporated herein by reference, U.S.S.R. Pat. No. 166,999, incorporated herein by reference, Federal German Patent No. 230,696, incorporated herein by reference and European Patent No. 158,336, incorporated herein by reference.

All of the devices described above substitute the finger of the physician with an inanimate probe. For this reason, they have an inherent drawback in that they remove certain advantages to the physician and the patient inherent in the touch of the physician's finger. The physician's finger is capable of determining with accuracy the precise point where the pain threshold is to be assessed. It is not always easy for the physician to press an inanimate probe at precisely the right location because he or she receives no direct tactile feedback from a probe. In addition, there is an impersonal aspect that is objectionable to some patients associated with the act of being pressed with an inanimate object. Many patients would prefer the more personal contact of a physician's finger.

In an attempt to overcome the above deficiencies, a dolorimeter was develop for pressure measurement during manual palpitation as described in U.S. Pat. No. 5,012,817, incorporated herein by reference and Canadian Patent No. 1,359,224, incorporated herein by reference. The dolorimeter uses a thin pressure sensor placed on a practitioner's finger so that the sense of touch associated with conventional palpation can be retained in combination with an objective measurement of the applied pressure. The pressure sensor provides an indication of the applied pressure based on a resistance change.

While U.S. Pat. No. 5,012,817 and CA 1,359,224 overcome a number of the deficiencies of the prior art, the invention taught in these patents also has deficiencies. For example, the practitioner must connect the pressure sensor to a separate read-out unit and keep track of the observed pressures. The read-out unit is typically wrist mounted. Further, the read-out unit is large and cannot form an integrated unit with the pressure sensor. In addition, communication between the pressure sensor and the read-out is electronic and requires that the two be hard wired to one another. The read-out requires that the practitioner observe the read out unit directly, removing attention from the patient. If the unit requires re-calibration, it must be returned to the factory for calibrating. Additionally, the pressure sensors have to be selected to meet strict specified pressure limits. This leads to increased production costs as there is a relatively high discard rate. It is an objective of the present invention to overcome the deficiencies in the prior art.

### SUMMARY

In a representative example, a dolorimeter is provided for assessing in a quantitative manner the pressure applied to a patient. The dolorimeter is an integral unit with a pressure/force sensing means and a reporting means being mountable on a practitioner's hand. The pressure/force sensing means is configured to be located between a practitioner's hand and the patient and is in communication with an integrally mounted reporting means. The sensor is sized and is of suitably selected material to promote substantial tactile contact between the hand of the practitioner and the patient so as to allow the practitioner to monitor manually the application and location of pressure or force applied.

In use, the dolorimeter is mounted on the practitioner's thumb or finger and the practitioner then palpates the patient. At the same time, the practitioner observes the patient, looking for signs of discomfort or listens for feedback from the patient. In the preferred embodiment of the invention, there is an audible signal corresponding to a level of pressure applied. The practitioner is able to record the level of pressure applied that results in pain or discomfort. Thus, over a series of appointments, the practitioner is able to reliably ascertain whether the pain is increasing, decreasing or remaining the same for a given patient.

In a second representative example, the dolorimeter is set to a predetermined pressure level and reports when that level of pressure has been achieved. Again the practitioner palpates the patient, but rather than waiting for feedback from the patient and then recording the level of pressure exerted, the dolorimeter is used to assist the practitioner is applying a predetermined level of pressure. The practitioner then observes whether or not there are signs of discomfort at that level of pressure.

In one embodiment of the invention the pressure/force sensing means is a pressure/force sensor.

In another aspect of the invention, the pressure/force sensor is comprised of a pressure/force sensing film.

In one aspect of the invention, the pressure/force sensor is a resistance sensor.

In another aspect of the invention, the pressure/force sensor is a capacitance sensor.

In another aspect of the invention, the pressure/force sensor is an inductance sensor.

In another aspect of the invention, the pressure/force sensor is an optical sensor.

In another aspect of the invention, the pressure/force sensor is an acoustic sensor.

In one embodiment of the invention an electronic means is provided for communication between the pressure/force sensor and the reporting means.

In one aspect of the invention, a resistance-to-voltage converter is provided to measure an output from the pressure/force sensor and communicate with the reporting means.

In another aspect of the invention, a suitably selected electronic signal processor is provided to process data from the pressure/force sensor and communicate with the reporting means.

In one aspect of the invention, the suitably selected electronic signal processor comprises a microprocessor for providing said signal representing the pressure applied to said sensor to said reporter.

In one aspect of the invention, the suitably selected electronic signal processor comprises an analogue to digital converter for providing said signal representing the pressure applied to said sensor to said reporter.

In a further aspect of the invention an analogue to digital converter and a microprocessor are provided to measure an output from the pressure/force sensor and communicate with the reporter.

In another aspect of the invention, the microprocessor converts the output to a numerical pressure level.

In one embodiment of the invention, the reporting means is comprised of a sound emitting member.

In one aspect of the invention, the sound emitting member is a piezoelectric speaker.

In another embodiment of the invention, the reporting means is a liquid crystal display

In another aspect of the invention, the reporting means is a digital display.

In yet another aspect of the invention, the digital display is an liquid crystal display.

In another embodiment of the invention, the reporting means is a light emitting member.

In one aspect of the invention, the light emitting member is a light emitting diode.

In another aspect of the invention, the light emitting member is an infra red emitting light.

In another embodiment of the invention, the reporting means is a radio wave emitting member.

In yet another embodiment of the invention there is provided means for calibrating the pressure/force sensing means with a suitably selected calibrator.

In one aspect of the invention, the means for calibrating the pressure/force sensing means is further provided with an initiator means to signal that calibration may commence.

In yet another aspect of the invention, the initiator means is an electronic initiator means.

In yet another aspect of the invention, the initiator is further defined as being a magnet-responsive initiator means.

In yet another aspect of the invention, there is a power management centre for operative coupling to the microprocessor.

In yet another aspect of the invention, there is provided a calibration table memory for operative coupling to the microprocessor.

In yet another aspect of the invention there is provided a programme memory for operative coupling to the microprocessor.

In one aspect of the invention the dolorimeter further comprises a ring-like band for fitting around the practitioner's digit.

In another aspect of the invention, the dolorimeter further comprises a closable strip for fitting around the practitioner's digit.

In another aspect of the invention a method of quantifying pressure applied to a patient by a practitioner using a dolorimeter is provided, comprising releasably retaining the dolorimeter on the practitioner's hand, wherein the dolorimeter is suitably selected to promote substantial tactile communication between the hand of the practitioner and the patient, palpating the patient with the dolorimeter, manually determining the application and location of pressure or force applied and measuring the pressure applied to the patient.

In another aspect of the invention the method comprises releasably retaining the dolorimeter on a finger of a practitioner.

In yet another aspect of the invention, the method comprises releasably retaining said dolorimeter on an index finger.

In yet another aspect of the invention, the method further comprises setting the dolorimeter at a predetermined pressure/force setting prior to palpating.

In yet another aspect of the invention, the initiator means is activated by an external magnetic field.

### BRIEF DESCRIPTION OF THE DRAWINGS

The advantages of the invention will be better understood with reference to the following drawings.

FIG. 1. is an side view of a first embodiment in accordance with the invention showing the dolorimeter on a practitioner's finger.

FIG. 2 is an isometric view of the first embodiment, shown in FIG. 1, in accordance with the invention.

FIG. 3 is a cross section longitudinal view of the dolorimeter of FIG. 1.

FIG. 4 is a schematic view of the circuitry of the dolorimeter of FIG. 1 in accordance with the invention.

FIG. 5 is an isometric view of a second embodiment in accordance with the invention.

### DETAILED DESCRIPTION

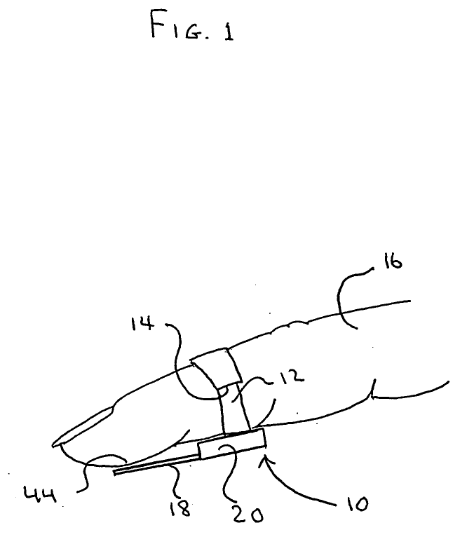

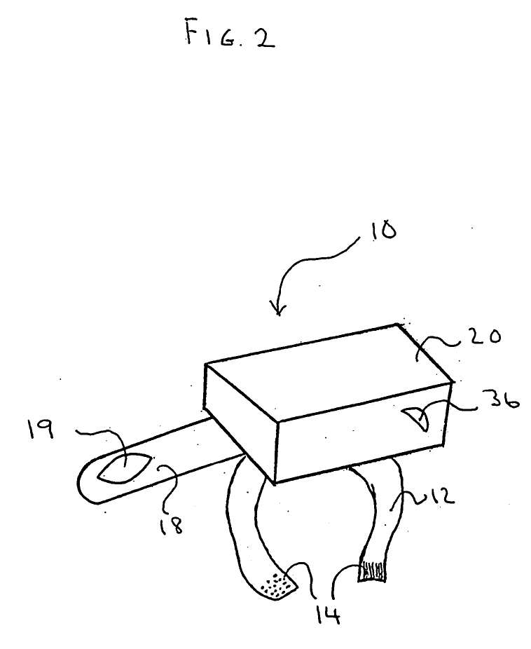

As shown in FIG. 1, a dolorimeter, generally referred to as 10 has a strip 12 with a closure 14 for closing the strip 12, such as Velcro® around a practitioner's finger or thumb (digit) 16, a pressure/force sensor support 18 and a housing 20. As shown in FIG. 2, the pressure/force sensor support 18 has at an end distal to the housing 20, a pressure/force sensor 19. The electrical resistance of the pressure/force sensor 19 varies according to pressure applied to the sensor 19.

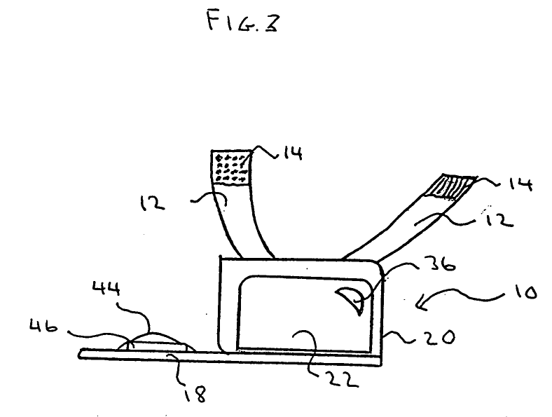

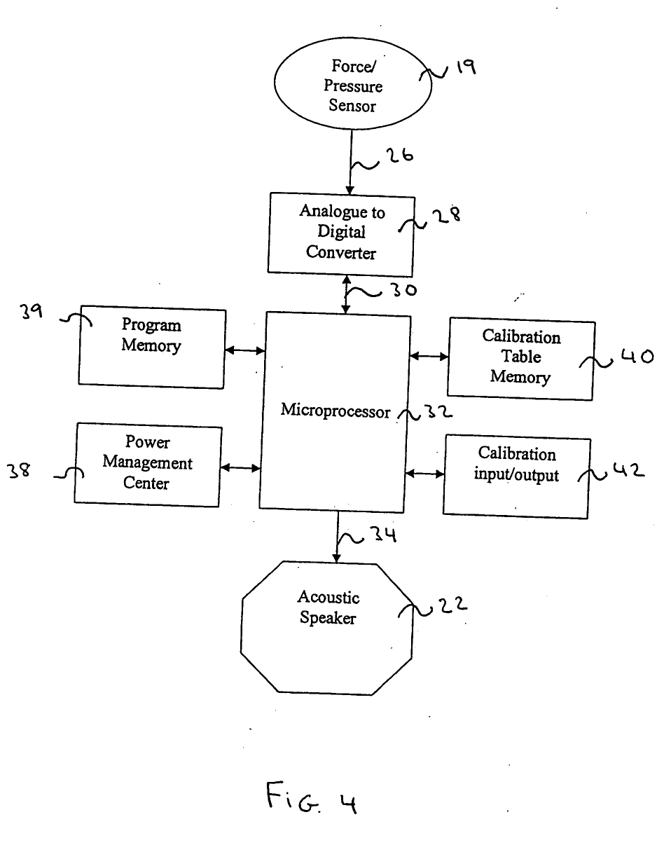

As shown in FIG. 3, the housing 20 retains a reporter 22 and is coupled with the pressure/force sensor support 18 to provide an integral apparatus for palpating a patient (not shown). As shown in FIG. 4 the pressure/force sensor 19 is in electronic communication with the reporter 22, as follows: a first electrical connection 26 connects the pressure/force sensor 19 to an analogue to digital converter 28, a second electrical connection 30 connects the analogue to digital converter 28 to a microprocessor 32, and a third electrical connection 34 connects the microprocessor 32 to the reporter 22, which in one embodiment is a piezoelectric speaker 36 (see FIGS. 2 and 3). A power management centre 38 is provided to automatically turn off the dolorimeter 10 after a programmed period of time. Calibration and programme memory capabilities are provided by a program memory 39, a calibration table memory 40 and calibration input/output 42, respectively, in electronic communication with the microprocessor 32.

In one embodiment, the pressure measured by the pressure/force sensor 19 is represented as short duration tones (beeps), or a continuous tone, or synthesized human voice emitted from the reporter 22. The beeps are generated in real time at the moment the pressure exceeds one of the predetermined pressure levels. The indication of pressure level (readings) can be accomplished by suitable acoustic means such as the number of beeps, the amplitude or duration of beeps, the frequency of beeps, the tone of beeps, or any combination thereof.

Referring now to the pressure/force sensor 19, it includes a pressure sensitive film 44 on a flexible, sheet-like substrate 46 as shown in FIG. 3. The film 44 is a shunt-mode force sensing resistor of the type sold by Interlink Electronics of Santa Barbara Calif., U.S.A. Force sensing resistors as supplied by the said company are in accordance with one or more of the following U.S. Pat. Nos: 4,451,714; 4,276,538; 4,314,228; 4,301,337; which are incorporated herein by reference. This device has an electrical resistivity which varies according to the pressure applied to the film 44. The film 44 is mounted on a substrate 46 which may be, for example, a relatively tin elastomeric membrane having contacts printed thereon for contacting the film 44. When the dolorimeter 10 is fitted in place, the film 44 is under tip 48 of the index finger 16 (see FIG. 1).

Referring no w to the analogue to digital converter 28 and the microprocessor 32, the analogue to digital converter 28 measure the resistance of the pressure/force sensor 19 and provides an output voltage signal representing resistance measured at the pressure/force sensor 19 and therefore ultimately represents the pressure applied to the pressure/force sensor 19. The microprocessor 32 has a novel pressure levels scale used is logarithmic according to Weber's Law but it can be customized. The number of pressure levels (resolution) used is 10, but the resolution can be different for a specific application. In some applications only a certain single pressure threshold might be required.

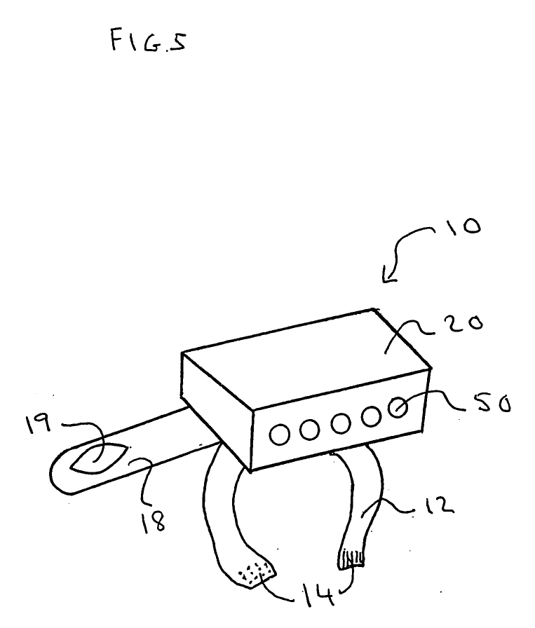

In a second embodiment, as shown in FIG. 5, several small light emitting diodes 50 or LED's display the pressure levels instead of an audible report. Using a combination of “on” LEDs one can reduce the number of LEDs for a given resolution. For instance, one LED can indicate lower or upper range and additional three LEDs can indicate a pressure level at a given range. This will allow distinguishing up to 6 levels with only four LEDs.

In another embodiment of the invention, there is a communications ability between the dolorimeter 10 and a computer (not shown) to record measured data. The dolorimeter 10 has a short range telemetry link, provided as an infrared light source (not shown) similar to that used for TV controls. Similarly communication between the dolorimeter 10 and a computer may.,be accomplished in yet another embodiment wherein a radio transmitter (not shown) is integrated into the dolorimeter 10.

There are two modes of operation. In both modes the practitioner may place the dolorimeter 10 on their hand in any convenient location, but preferably on a digit, and more preferably on an index finger 16. The dolorimeter 10 is releasably retained by a closeable strip 12 that forms a ring about the finger 16 as shown in FIG. 1. When in place, the sensor 19 is located on the tip 48 of the finger 16. The dolorimeter 10 is switched to the on position and the practitioner then palpates the patient. In the first mode, the reporter 22 reports in real time. As the practitioner gradually applies pressure to the patient, the resistance of the pressure/force sensor 19 changes. This change is transmitted by means of the microprocessor 32 to the reporter 22. At the onset of pain indicated by the patient, the practitioner releases the pressure level and records the pressure level applied. Using an alternative embodiment of the invention, there is provided means for data collection and therefore, the practitioner may collect data for downloading to a computer or other suitably selected data processor at a later time. Independent of the method of data collection, the practitioner is able to monitor the patient's response to pressure and determine whether the response is changing over the treatment/observation time, thereby arriving at a quantitative assessment of pain or discomfort. The dolorimeter 10 automatically shuts off a few minutes after usage. This is effected by the power management centre 38.

The second mode of operation utilizes preset pressure/force levels. The practitioner sets the pressure level then palpates the patient at that pressure level. The dolorimeter 10 reports when the preset level is achieved. In this manner, the practitioner can routinely palpate at a given pressure level and ascertain whether that pressure causes an increase or decrease in pain, or whether the pain level remains the same over the duration of the treatment/observation time. As with the first mode of operation, the dolorimeter 10 automatically shuts off a few minutes after usage.

Regardless of the mode of operation, the dolorimeter 10 should be calibrated with a suitably selected calibrator (not shown). A method for calibrating is as follows: A. magnet (or an electromagnet) is brought close to the dolorimeter 10 which closes a reed switch on the dolorimeter for 15 seconds. This closes an input pin on the microprocessor 32 and requests calibration mode. After several (preprogrammed) seconds the dolorimeter 10 beeps, signaling that it has entered calibration mode. The magnet is pulled away.

2) The pressure on the sensor 19 by the calibrator is set to the first sensitivity setting of the dolorimeter 10 (usually 100 grams) and the magnet is pulled down momentarily. This closes the reed switch and signals the microprocessor 32 that the pressure reading from the sensor 19 is equivalent to, for example, 100 grams. The dolorimeter 10 beeps in acknowledgement. The magnet is pulled away.

3) Step 2 is repeated as many more times as there are levels of force/pressure readings on the dolorimeter 10. After the last step, the dolorimeter 10 beeps 5 times signaling the end of the calibration process. The dolorimeter 10 is taken out from the calibrator and is ready for use.

As the force exercised by the examining finger 16 can be different for different fingers even with the same pressure as sensed by the pressure/force sensor 19, the practitioner may want to calibrate the sensor 19 according to force applied by a specific examining finger 16 rather than the pressure. This can be accomplished by monitoring of the force applied by the finger 16 on the sensor 19 and by calibrating the dolorimeter 10 at desired force levels rather than pressure levels. This can be done by placing the sensor 19 of the dolorimeter 10 on a force measuring scale and gradually increasing the force exercised by the examining finger 16. Once the desired force level is achieved, the dolorimeter 10 is automatically calibrated for that level. For instance the selected force levels for resolution of 5 levels can be 200 gf, 400 gf, 800 gf, 1600 gf and 3200 gf. Calibration can also be carried out by the practitioner.

While specific embodiments of the invention have been described, such embodiments should not be considered as limiting the scope of the invention as construed in accordance with the accompanying claims. For example, the tactile communication in the embodiment described is achieved by making the film and the substrate relatively thin to accommodate the sense of the touch of the practitioner. Other possibilities include providing a thin substrate with a very small, but rigid pressure sensitive unit which would be relatively unobtrusive—again permitting substantial tactile communication between the practitioner and the patient.

In clinical examples, forces are applied to patients or other subjects by physicians, physical therapists, or other practitioners, but evaluation of a subject can carried out by a subject's family member, friend, or other evaluator. In addition, the subject can act as both subject and evaluator.

In some illustrated examples, the pressure/force sensor 19 and a display or other reporter 22 are fixed to the dolorimeter housing 20. In other examples, the pressure/force sensor 19 and the reporter 22 are separate and can be individually fixed to, for example, a digit 16 of an evaluator so that the sensor 19 and the reporter 22 are situated in a common visual field of the evaluator. For example, a pressure/force sensor 19 can be attached to an evaluator's digit 16 using a first strap (such as a VELCRO™ strap), and the reporter 22 can be attached to the evaluator's digit 16 using a second strap. A flexible or rigid electrical interconnection between the sensor 19 and the reporter 22 can be provided. Typically a processor is provided to receive an electrical signal from the sensor 19 and associated with a force or pressure applied to the sensor 19. The processor can include a microprocessor 32, an analogue to digital converter 28, and a buffer amplifier configured to receive and condition the sensor electrical signal. While the processor can be integrated into a common housing with the reporter 22 and to which the sensor 19 can be fixed, the processor can be configured to be situated at another location such as, for example, on the evaluator's arm, a second digit of the evaluator, or on the evaluator's belt or clothing.

In examples in which a reporter 22 provides an audible indication of an applied force, the reporter can be situated at a variety of locations. For example, the sensor 19 can be fixed to an evaluator's digit and the reporter situated on the evaluator's arm, waist, or other location. Alternatively, an earphone or headphone can be provided so that the subject is unaware of the audible indication.

1. A dolorimeter for quantifying a pressure or a force applied to a patient by a hand of a practitioner, said dolorimeter being an integral unit comprising:
a housing for coupling to a suitably selected releasable retention means;
a pressure/force sensor having a property which varies according to pressure or force applied to said sensor, said sensor for coupling to said housing, the sensor being sized and of suitably selected material to promote substantial tactile communication between the hand of the practitioner and the patient so to allow the practitioner to monitor manually the application and location of pressure or force applied; and
a reporter coupled to said housing, said reporter for electronic communication with said pressure/force sensor, such that in use, the pressure or force applied by the practitioner's hand is reported as a quantified output.

2. The dolorimeter of claim 1, more specifically for quantifying a pressure or a force applied to a patient by a digit of a practitioner, wherein said sensor is sized and of suitably selected material to promote substantial tactile communication between the digit of the practitioner and the patient.

3. The dolorimeter of claim 1, wherein said pressure/force sensor is an electrical sensor selected from the group consisting of a resistance sensor, a capacitance sensor and an inductance sensor.

4. The dolorimeter of claim 1, wherein said pressure/force sensor is a fibre optic sensor.

5. The dolorimeter of claim 4, wherein the pressure/force sensor is a pressure sensitive film.

6. The dolorimeter of claim 5, wherein the pressure sensitive film is on a flexible substrate.

7. The dolorimeter of claim 6, further comprising a releasable retention means coupled to said dolorimeter, for releasably retaining said dolorimeter from the hand of the practitioner.

8. The dolorimeter of claim 2, further comprising a releasable retention means coupled to said dolorimeter forming a band sized to fit the practitioner's digit.

9. The dolorimeter of claim 8, further comprising a suitably selected electronic signal processor for operable communication with said pressure/force sensor and said reporter.

10. The dolorimeter of claim 9, wherein said suitably selected electronic signal processor is selected to quantify the force or pressure exerted on said sensor for providing a signal representing the pressure or force applied to the member.

11. The dolorimeter of claim 10, wherein said suitably selected electronic signal processor comprises a microprocessor for providing said signal representing the pressure applied to said sensor to said reporter.

12. The dolorimeter of claim 11, further comprising an analogue to digital converter for providing said signal representing the pressure applied to said sensor from said analogue to digital converter to said reporter.

13. The dolorimeter of claim 12, wherein said suitably selected electronic signal processor is comprised of means for tracking said signal and for holding automatically said signal in a state representing a highest value of pressure or force applied to said sensor.

14. The dolorimeter of claim 13, wherein said means for tracking and holding is reset when said signal crosses a threshold value.

15. The dolorimeter of claim 14, wherein said reporter has a digital display.

16. The dolorimeter of claim 14, wherein said reporter has a light emitting display.

17. The dolorimeter of claim 14, wherein said reporter is a sound emitting reporter.

18. The dolorimeter of claim 17, wherein said sound emitting reporter is a piezoelectric speaker.

19. The dolorimeter of any one of claims 1 to 14 claims 14, wherein said reporter is a radio wave emitting reporter.

20. The dolorimeter of claim 19, further comprising a power management circuit for operative communication with said microprocessor.

21. The dolorimeter of claim 21, further comprising a calibration table memory for operative communication with the microprocessor.

22. The dolorimeter of claim 21, further comprising an initiator for signaling that calibration of said calibrating table memory may proceed.

23. The dolorimeter of claim 22, wherein the initiator is an electronic initiator.

24. The dolorimeter of claim 23, wherein said initiator is selected to be responsive to an external magnetic force.

25. The dolorimeter of claim 24, further comprising a programme memory for operative communication with the microprocessor.

26. A dolorimeter for quantifying a pressure or a force applied to a patient by a digit of a practitioner, said dolorimeter being an integral unit comprising:
a housing for coupling to a suitably selected releasable retention means;
a pressure/force sensor having an electrical resistance which varies according to pressure or force applied to said sensor, said sensor for coupling to said housing, the sensor being sized and of suitably selected material to promote substantial tactile communication between the digit of the practitioner and the patient so to allow the practitioner to monitor manually the application and location of pressure or force applied;
a reporter coupled to said housing, said reporter for electronic communication with said pressure/force sensor; and
a suitably selected electronic processor for operative communication with said sensor and said reporter such that in use, the pressure or force applied by the practitioner's digit is reported as a quantified output.

27. A method of quantifying pressure applied to a patient by a practitioner using a dolorimeter comprising;
releasably retaining said dolorimeter on the practitioner's hand, said dolorimeter being suitably selected to promote substantial tactile communication between the hand of the practitioner and the patient;
palpating the patient with said dolorimeter;
manually determining the application and location of pressure or force applied; and
measuring the pressure applied to the patient.

28. The method of claim 27, comprising releasably retaining said dolorimeter on a finger of a practitioner.

29. The method of claim 28, comprising releasably retaining said dolorimeter on an index finger.

30. The method of any claim 29, further comprising setting the dolorimeter at a predetermined pressure/force setting prior to palpating.

31. The method of claim 30, further comprising calibrating said dolorimeter with a suitably selected calibrator prior to use.

32. The method of claim 31, wherein calibration is initiated by an initiator means.

33. A dolorimeter for assessing a force or pressure applied to an evaluation region by an evaluator, comprising:
a sensor responsive to the applied force and configured to provide substantial tactile communication between the evaluator and the evaluation region;
a reporter in communication with the sensor and configured to produce an indication associated with the applied force; and
a retainer configured to fix the sensor and the reporter to the evaluator so that the sensor and the reporter are within a common visual field of the evaluator.

34. The dolorimeter of claim 33, further comprising a housing, wherein the sensor and the reporter are fixed to the housing.

35. The dolorimeter of claim 34, wherein the retainer is configured to fix the housing to a digit of the evaluator.

36. The dolorimeter of claim 33, wherein the retainer is configured to fix the sensor and the reporter to a digit of the evaluator.

37. The dolorimeter of claim 33, further comprising a processor configured to produce an electrical signal associated with the applied force and to deliver the produced electrical signal to the reporter.

38. The dolorimeter of claim 37, wherein the reporter includes a numerical display configured to display a numeric value associated with the applied force.

39. The dolorimeter of claim 37, wherein the reporter includes at least one visual indicator activatable by the processor based on the applied force.

40. The dolorimeter of claim 39, wherein the at least one visual indicator includes at least one light emitter.

41. The dolorimeter of claim 39, wherein the at least one visual indicator includes at least one light emitter associated with a first color and at least one light emitter associated with a second color.

42. The dolorimeter of claim 37, wherein the processor is configured to reset the reporter if the applied force is less than a predetermined reset value.

43. The dolorimeter of claim 33, wherein the reporter is configured to indicate that the applied force exceeds a predetermined value.

44. A dolorimeter for assessing a force or pressure applied to an evaluation area of a subject by an evaluator, comprising:
a sensor responsive to an applied force and configured to provide substantial tactile communication between the evaluator and a portion of the subject to which the force is applied;
an audio reporter in communication with the sensor and configured to produce an audio indication of the applied force; and
a retainer configured to fix the sensor to the evaluator so that the sensor is situated between the evaluation area and the evaluator.

45. A method of quantifying a force applied to a subject by an evaluator, comprising;
applying a force to an evaluation area of the subject with a hand of the evaluator;
assessing a magnitude of the applied force based on a force sensor situated to promote substantial tactile communication between the hand of the evaluator and the evaluation area; and
obtaining an estimate of the assessed applied force magnitude based on a reporter situated with a visual field that includes the evaluation area.

46. The method of claim 45, wherein the sensor is fixed to a digit of the evaluator.

47. The dolorimeter of claim 16, wherein the at least one light emitting display includes at least one light emitter associated with a first color and at least one light emitter associated with a second color.
